(19) United States
(12) Patent Application Publication
(10) Pub. No.: US 2021/0134462 A1
(43) Pub. Date: May 6, 2021

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Takeshi FUKUDA, Kanagawa (JP); Haruyasu NAKATSUGAWA, Kanagawa (JP)

(21) Appl. No.: 17/072,039

(22) Filed: Oct. 16, 2020

(30) Foreign Application Priority Data
Oct. 31, 2019 (JP) 2019-199068

(51) Int. Cl. G16H 50/20; G16H 10/60; G16H 50/50; G16H 40/20; G16H 70/60; G16H 50/30; A61D 99/00

(52) U.S. Cl. CPC G16H 50/20; G16H 10/60; G16H 50/50; A61D 99/00; G16H 70/60; G16H 50/30; G16H 40/20

(57) ABSTRACT

Provided are an information processing apparatus, an information processing method, and an information processing program capable of suppressing an animal from being affected with a susceptible disease. The information processing apparatus derives, on the basis of state information representing a state of a subject that is a basis of diagnosis of an animal that is the subject and a breed of the subject, a degree of risk that the subject is affected with a susceptible disease; determines whether or not notification of a message for promoting visit to a veterinary hospital is necessary on the basis of the degree of the risk; and notifies an owner of the subject of the message in a case where it is determined that notification of the message is necessary.

### CROSS-REFERENCE TO RELATED APPLICATION

This application claims priority under 35 USC 119 from Japanese Patent Application No. 2019-199068 filed Oct. 31, 2019, the disclosure of which is incorporated by reference herein.

### BACKGROUND

### Technical Field

The present disclosure relates to an information processing apparatus, an information processing method, and an information processing program.

### Related Art

There is disclosed a technique of transmitting a message for preventing occurrence of subjective symptoms of a user to a user's terminal using information of the user who is a human and environmental factors (see JP2015-219700A).

### SUMMARY

By the way, in a case where a subject is an animal, a susceptible disease of the animal differs depending on an animal breed. The term “animal breed” as used herein means an animal type that is a subdivision of a race of an animal such as a dog and a cat, and includes classification by animal breeds such as Chihuahua, classification by head species such as short-headed breeds, classification by sizes such as large dogs, and the like.

However, the technique disclosed in JP2015-219700A is intended for humans, and does not consider preventing an animal from being affected with a susceptible disease in consideration of a breed of an animal.

The present disclosure has been made in view of the above circumstances, and provides an information processing apparatus, an information processing method, and an information processing program capable of suppressing an animal from being affected with a susceptible disease.

In order to achieve the above object, according to an aspect of the present disclosure, there is provided an information processing apparatus comprising: a derivation unit that derives, on the basis of state information representing a state of a subject that is a basis of diagnosis of an animal that is the subject and a breed of the subject, a degree of risk that the subject is affected with a susceptible disease; a determination unit that determines whether or not notification of a message for promoting visit to a veterinary hospital is necessary on the basis of the degree of the risk; and a notification unit that notifies an owner of the subject of the message in a case where it is determined by the determination unit that the notification of the message is necessary.

In the information processing apparatus according to the aspect of the present disclosure, the notification unit may notify the owner of the subject of the message at a higher frequency as the degree of the risk is higher in a case where it is determined by the determination unit that the notification of the message is necessary.

In addition, in the information processing apparatus according to the aspect of the present disclosure, the derivation unit may derive the degree of the risk using a future prediction result of the state information in addition to the state information up to the present.

Further, in the information processing apparatus according to the aspect of the present disclosure, the state information may include at least one of information obtained by examining the subject or an examination result of the subject.

Further, according to another aspect of the present disclosure, there is provided an information processing method executed by a computer, the method comprising: deriving, on the basis of state information representing a state of a subject that is a basis of diagnosis of an animal that is the subject and a breed of the subject, a degree of risk that the subject is affected with a susceptible disease; determining whether or not notification of a message for promoting visit to a veterinary hospital is necessary on the basis of the degree of the risk; and notifying an owner of the subject of the message in a case where it is determined that the notification of the message is necessary.

Further, according to still another aspect of the present disclosure, there is provided an information processing program causing a computer to execute: a process of deriving, on the basis of state information representing a state of a subject that is a basis of diagnosis of an animal that is the subject and a breed of the subject, a degree of risk that the subject is affected with a susceptible disease; a process of determining whether or not notification of a message for promoting visit to a veterinary hospital is necessary on the basis of the degree of the risk; and a process of notifying an owner of the subject of the message in a case where it is determined that the notification of the message is necessary.

Further, according to still another aspect of the present disclosure, there is provided an information processing apparatus comprising: a memory that stores a command executed by a computer; and a processor configured to execute the stored command, in which the processor derives, on the basis of state information representing a state of a subject that is a basis of diagnosis of an animal that is the subject and a breed of the subject, a degree of risk that the subject is affected with a susceptible disease; determines whether or not notification of a message for promoting visit to a veterinary hospital is necessary on the basis of the degree of the risk; and notifies an owner of the subject of the message in a case where it is determined that the notification of the message is necessary.

According to the present disclosure, it is possible to suppress an animal from being affected with a susceptible disease.

### BRIEF DESCRIPTION OF THE DRAWINGS

Exemplary Embodiments of the present disclosure will be described in detail with reference to the following figures, wherein:

FIG. 1 is a block diagram showing an example of a configuration of an information processing system.

FIG. 2 is a block diagram showing an example of a hardware configuration of the information processing apparatus.

FIG. 3 is a diagram showing an example of electronic medical record data.

FIG. 4 is a block diagram showing an example of a functional configuration of the information processing apparatus.

FIG. 5 is a diagram for explaining a process of deriving the degree of risk that a subject is affected with a susceptible disease.

FIG. 6 is a diagram for explaining a process of deriving the degree of risk that a subject is affected with a susceptible disease.

FIG. 7 is a diagram showing an example of a visit promotion message.

FIG. 8 is a diagram for explaining a notification timing.

FIG. 9 is a diagram for explaining a notification timing.

FIG. 10 is a flowchart showing an example of a notification process.

FIG. 11 is a graph for explaining an example of using a prediction result.

### DETAILED DESCRIPTION

Hereinafter, an example for carrying out the technique of the present disclosure will be described in detail with reference to the accompanying drawings. It should be noted that in the following embodiments, an example in which a dog is applied as an animal that is a subject will be described. In addition, the term “animal” in the present specification means animals such as dogs and cats, excluding “humans”.

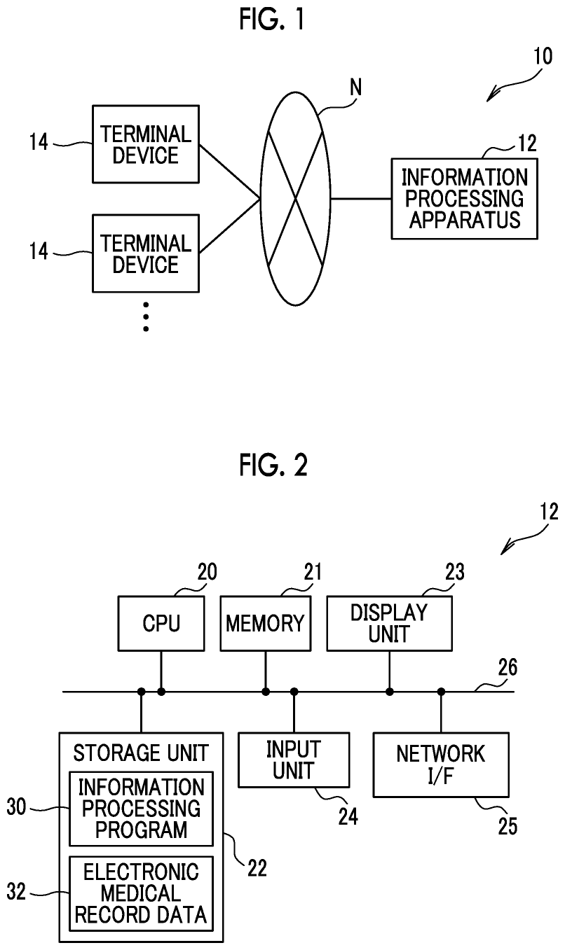

First, a configuration of an information processing system 10 according to the present embodiment will be described with reference to FIG. 1. As shown in FIG. 1, the information processing system 10 includes an information processing apparatus 12 and a plurality of terminal devices 14. The information processing apparatus 12 and the plurality of terminal devices 14 are respectively connected to a network N, and are able to communicate with each other through the network N.

The information processing apparatus 12 is provided in, for example, a veterinary hospital. Examples of the information processing apparatus 12 include a server computer and the like. The information processing apparatus 12 may be a cloud server. The terminal device 14 is, for example, a terminal device owned by an owner of an animal that is a subject. Examples of the terminal device 14 include a smartphone and the like.

Next, a hardware configuration of the information processing apparatus 12 according to the present embodiment will be described with reference to FIG. 2. As shown in FIG. 2, the information processing apparatus 12 includes a central processing unit (CPU) 20, a memory 21 as a temporary storage area, and a non-volatile storage unit 22. Further, the information processing apparatus 12 includes a display unit 23 such as a liquid crystal display, an input unit 24 such as a keyboard and a mouse, and a network interface (UF) 25 connected to the network N. The CPU 20, the memory 21, the storage unit 22, the display unit 23, the input unit 24, and the network OF 25 are connected to a bus 26.

The storage unit 22 is realized by a hard disk drive (HDD), a solid state drive (SSD), a flash memory, or the like. An information processing program 30 is stored in the storage unit 22 that is a storage medium. The CPU 20 reads out the information processing program 30 from the storage unit 22, develops the information processing program 30 in the memory 21, and executes the developed information processing program 30.

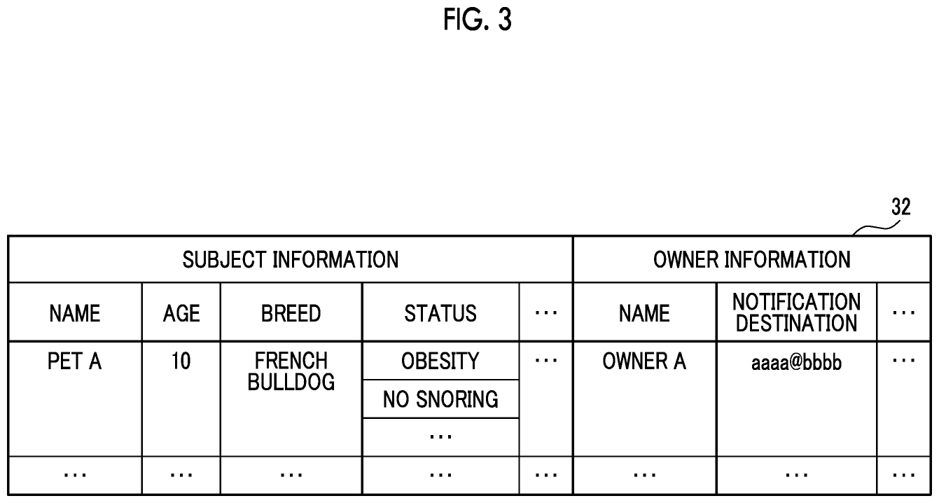

Further, the storage unit 22 stores electronic medical record data 32 including data used for an electronic medical record of a veterinary hospital. FIG. 3 shows an example of the electronic medical record data 32. As shown in FIG. 3, the electronic medical record data 32 includes subject information about an animal that is a subject and owner information about an owner of the subject animal associated with the subject information.

The subject information includes a name of a subject, an age of the subject, and a breed of the subject, as an example of identification information of the subject, and state information indicating a state of the subject that serves as a basis for diagnosing an animal that is the subject. The state information of the subject includes, for example, information obtained by examining the subject, through examination of the subject, palpation of the subject by a veterinarian, and inquiry by the veterinarian to an owner of the subject, for example. Further, the owner information includes a name of the owner and a notification destination of information to the owner, as an example of identification information of the owner. An e-mail address or the like may be given as an example of the notification destination of information for the owner.

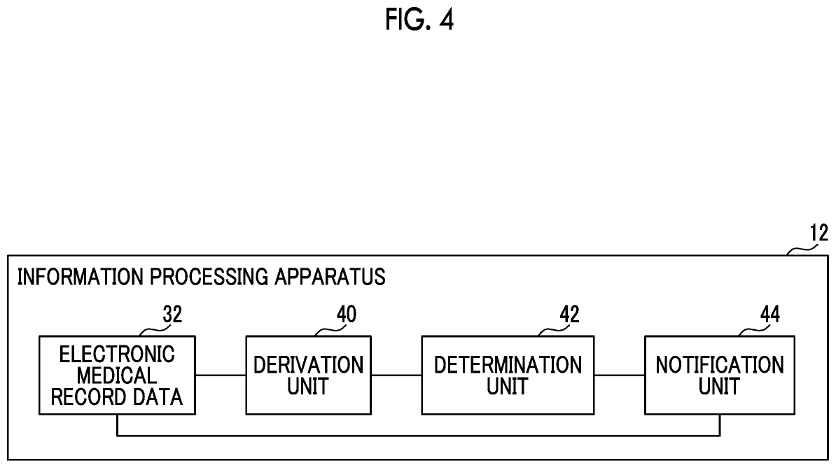

Next, a functional configuration of the information processing apparatus 12 according to the present embodiment will be described with reference to FIG. 4. As shown in FIG. 4, the information processing apparatus 12 includes a derivation unit 40, a determination unit 42, and a notification unit 44. The CPU 20 executes the information processing program 30 to function as the derivation unit 40, the determination unit 42, and the notification unit 44.

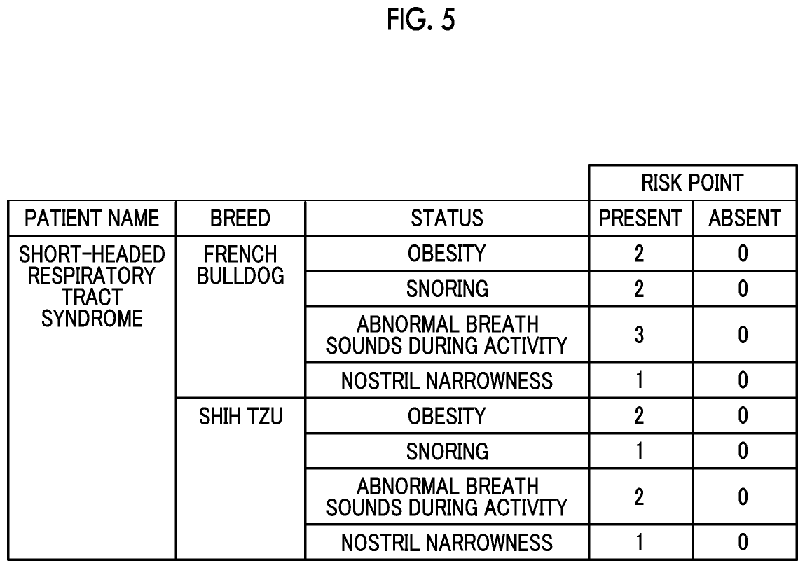

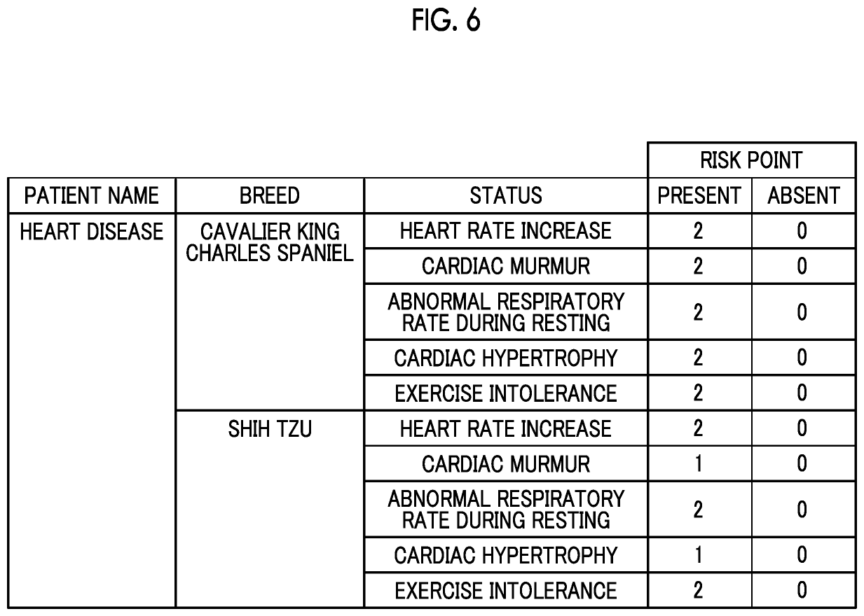

The derivation unit 40 derives the degree of risk (hereinafter referred to as a “risk degree”) that a subject is affected with a susceptible disease on the basis of state information of a subject and a breed of the subject, with reference to the electronic medical record data 32. Hereinafter, a specific example of the risk degree derivation process performed by the derivation unit 40 will be described with reference to FIGS. 5 and 6.

FIG. 5 shows an example in which French Bulldog and Shih Tzu are applied as breeds for which a disease of a risk degree derivation target is a short-headed respiratory tract syndrome and the short-headed respiratory tract syndrome is a susceptible disease. As shown in FIG. 5, in a case where the disease is the short-headed respiratory tract syndrome, the presence or absence of each of obesity, snoring, abnormal breath sounds during activity, and nostril narrowness is used as a state of a subject. In addition, as the state of the subject, risk points in a case where obesity, snoring, abnormal breath sounds during activity, and nostril narrowness are “present” are determined in advance. This risk point is set in advance for each combination of a disease and a breed having the disease as a susceptible disease, and a larger value is assigned to a breed having a higher risk of being affected with the disease. That is, in the example of FIG. 5, it is shown that French Bulldog has a higher risk of being affected with the short-headed respiratory tract syndrome than Shih Tzu.

FIG. 6 shows an example in which Cavalier King Charles Spaniel and Shih Tzu are applied as breeds for which a disease of a risk degree derivation target is a heart disease and the heart disease is a susceptible disease. As shown in FIG. 6, in a case where the disease is the heart disease, risk points in a case where heart rate increase, cardiac murmur, abnormal respiratory rate during resting, cardiac hypertrophy, and exercise intolerance are “present” are determined in advance as the state of the subject. In the example of FIG. 6, Cavalier King Charles Spaniel has a higher risk of being affected with the heart disease than Shih Tzu.

Further, in the present embodiment, “0” is assigned as the risk point in a case where each state of the subject is “absent”. The states of the subjects and the risk points used in FIGS. 5 and 6 are examples, and the present invention is not limited to these examples.

The derivation unit 40 derives a total value of risk points of a subject, for a disease of a risk degree derivation target, on the basis of state information of the subject of a breed having the disease as a susceptible disease and a risk point assigned for each state, with reference to the electronic medical record data 32.

For example, in a case where the disease is the short-headed respiratory tract syndrome, the breed of the subject is French Bulldog, the states of the subject of obesity and snoring are “present”, and abnormal breath sounds during activity and nostril narrowness are “absent”, a total value of risk points of the subject is 4 (=2+2+0+0). In addition, for example, in a case where the disease is the short-headed respiratory tract syndrome, the breed of the subject is Shih Tzu, the states of the subject of obesity, snoring and nostril narrowness are “present”, and abnormal breath sounds during activity is “absent”, a total value of risk points of the subject is 4 (=2+1+0+1).

Also, for example, in a case where the disease is the heart disease, the breed of the subject is Cavalier King Charles Spaniel, the states of the subject of heart rate increase, cardiac murmur, abnormal respiratory rate during resting, cardiac hypertrophy, and exercise intolerance are “present”, a total value of risk points of the subject is 10 (=2+2+2+2+2). Further, for example, in a case where the disease is the heart disease, the breed of the subject is Shih Tzu, the states of the subject of heart rate increase is “present”, and cardiac murmur, abnormal respiratory rate during resting, cardiac hypertrophy, and exercise intolerance are “absent”, a total value of risk points of the subject is 2 (=2+0+0+0+0).

Then, the derivation unit 40 derives a risk degree on the basis of the derived total value of the risk points. Specifically, in a case where the disease is the short-headed respiratory tract syndrome, the derivation unit 40 derives the risk degree as “none” in a case where the total value of the risk points is 0. Further, in this case, in a case where the total value of risk points is 1, the derivation unit 40 derives the risk degree as “low”. Further, in this case, in a case where the total value of the risk points is 2 or more and 3 or less, the derivation unit 40 derives the risk degree as “medium”. In addition, in this case, in a case where the total value of the risk points is 4 or more, the derivation unit 40 derives the risk degree as “high”.

In a case where the disease is the heart disease, the derivation unit 40 derives the risk degree as “none” in a case where the total value of the risk points is 0. Further, in this case, in a case where the total value of the risk points is 1 or more and 2 or less, the derivation unit 40 derives the risk degree as “low”. Further, in this case, in a case where the total value of the risk points is 3 or more and 5 or less, the derivation unit 40 derives the risk degree as “medium”. Further, in this case, in a case where the total value of the risk points is 6 or more, the derivation unit 40 derives the risk degree as “high”. As described above, in the present embodiment, four levels of “none”, “low”, “medium”, and “high” are applied as the number of risk degree levels, but the present invention is not limited thereto, and three or less levels may be used, or five or more levels may be used.

The determination unit 42 determines, on the basis of the risk degree derived by the derivation unit 40, whether or not notification of a message for promoting visit to a veterinary hospital (hereinafter, referred to as a “visit promotion message”) is necessary. In the present embodiment, in a case where the risk degree is “none”, the determination unit 42 determines that the notification of the visit promotion message is not necessary. On the other hand, in a case where the risk degree is “low”, “medium”, or “high”, the determination unit 42 determines that the notification of the visit promotion message is necessary.

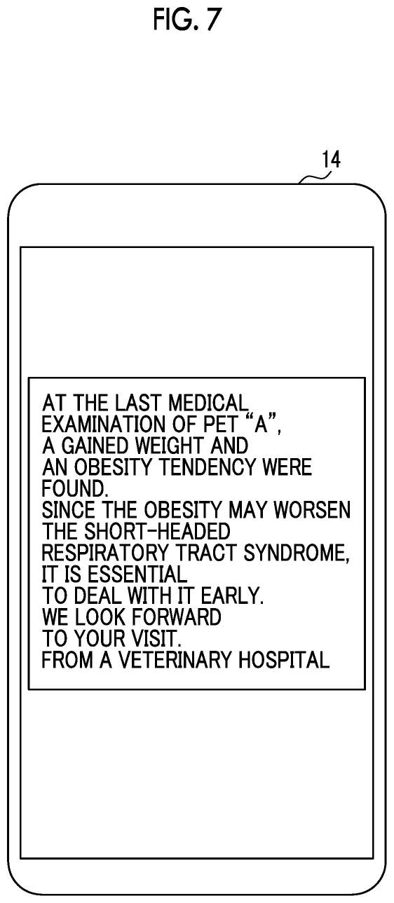

In a case where the determination unit 42 determines that the notification of the visit promotion message is necessary, the notification unit 44 notifies the owner of the subject of the visit promotion message. Specifically, the notification unit 44 notifies the owner of the subject of the visit promotion message by transmitting an e-mail to an e-mail address of a notification destination of the owner of the subject with reference to the electronic medical record data 32. By this notification, as shown in FIG. 7 as an example, the visit promotion message is displayed on a display unit of the terminal device 14 owned by the owner. The notification unit 44 may notify the visit promotion message through an application program such as a visit management application installed in the terminal device 14.

In addition, in a case where the determination unit 42 determines that the notification of the visit promotion message is necessary, the notification unit 44 notifies the owner of the subject of the visit promotion message at a higher frequency as the risk degree derived by the derivation unit 40 is higher. Specifically, for example, in a case where the disease is the short-headed respiratory tract syndrome, the notification unit 44 notifies the owner of the subject of the visit promotion message once every three months in a case where the risk degree is “low”, once every two months in a case where the risk degree is “medium”, and once every month in a case where the risk degree is “high”. In the present embodiment, the frequency according to the risk degree is determined in advance for each disease.

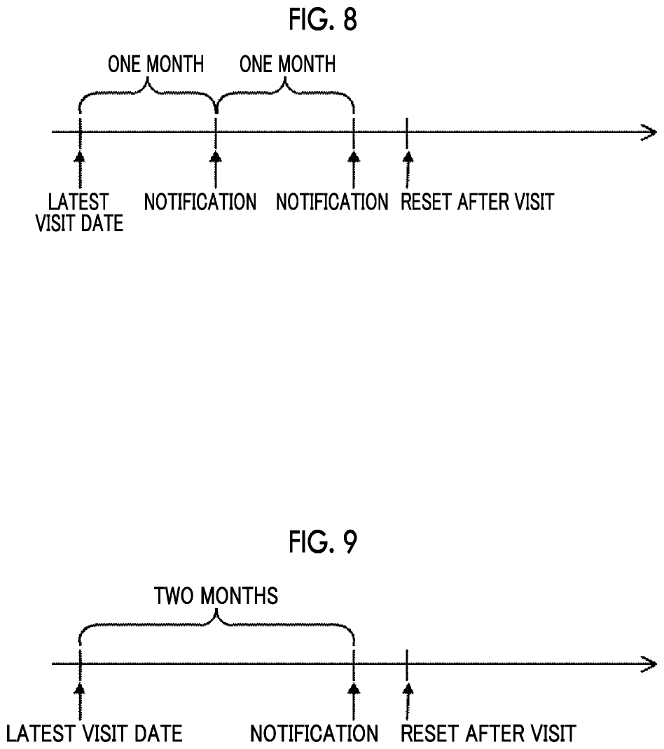

Accordingly, as shown in FIG. 8, in a case where the frequency is once a month, the visit promotion message is notified in a case where one month has passed from a later date among the latest visit date and the latest notification date. Similarly, as shown in FIG. 9, in a case where the frequency is once every two months, the visit promotion message is notified in a case where two months have passed from a later date among the latest visit date and the latest notification date.

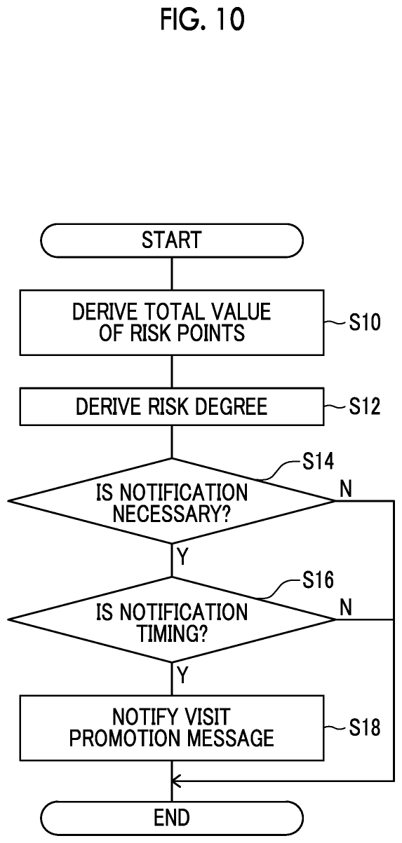

Next, an operation of the information processing apparatus 12 according to the present embodiment will be described with reference to FIG. 10. A notification process shown in FIG. 10 is executed as the CPU 20 executes the information processing program 30. The notification process shown in FIG. 10 is executed at a regular timing, for example, once a day. Further, the notification process shown in FIG. 10 is executed for each of the subjects having a target disease as a susceptible disease.

In Step S10 of FIG. 10, as described above, the derivation unit 40 derives a total value of risk points of a subject, with reference to the electronic medical record data 32, for a disease for which the risk degree is to be derived, on the basis of state information of the subject of a breed having the disease as a susceptible disease and risk points assigned to each state. In Step S12, the derivation unit 40 derives the risk degree on the basis of the total value of the risk points derived in Step S10, as described above.

In Step S14, the determination unit 42 determines, as described above, whether or not notification of a visit promotion message is necessary on the basis of the risk degree derived in Step S12. In a case where this determination is affirmative, the process proceeds to Step S16.

In Step S16, as described above, the notification unit 44 determines whether or not it is time to notify the visit promotion message at a frequency according to the risk degree derived in Step S12. In a case where this determination is affirmative, the process proceeds to Step S18. In Step S18, the notification unit 44 notifies the owner of the subject of the visit promotion message, as described above.

In a case where the process of Step S18 ends, the notification process ends. Further, in a case where the determination in Step S14 is negative, the notification process ends without executing the processes of Steps S16 and S18. In addition, in a case where the determination in Step S16 is negative, the notification process ends without executing the process of Step S18.

As described above, according to the present embodiment, the degree of risk that a subject is affected with a susceptible disease is derived on the basis of state information indicating a state of the subject that is a basis of diagnosis of an animal that is the subject and a breed of the subject. Then, it is determined whether or not the notification of a message for promoting visit to a veterinary hospital is necessary on the basis of the derived risk degree, and in a case where it is determined that the notification is necessary, the message is notified to an owner of the subject. Thus, it is possible for the owner of the subject to know at a proper timing that the subject should be examined at a veterinary hospital, and as a result, it is possible to suppress the animal that is the subject from being affected with the susceptible disease.

In addition, in the above embodiment, a case where information obtained by examining a subject is applied as state information representing a state of the subject which is a basis of diagnosis of an animal that is the subject has been described, but the present invention is not limited thereto. For example, a configuration in which an examination result of a subject obtained by examining the subject is applied as the state information may be used. The examination result in this case may include, for example, a total cholesterol value and a glucose level obtained by examining the blood of the subject. In this case, for example, the derivation unit 40 derives the risk degree as “none” in a case where the examination result is within a range of normal values. Further, in this case, for example, in a case where the examination result is within a range of abnormal values, the derivation unit 40 derives the risk degree as a higher degree as a deviation amount from the normal values is larger.

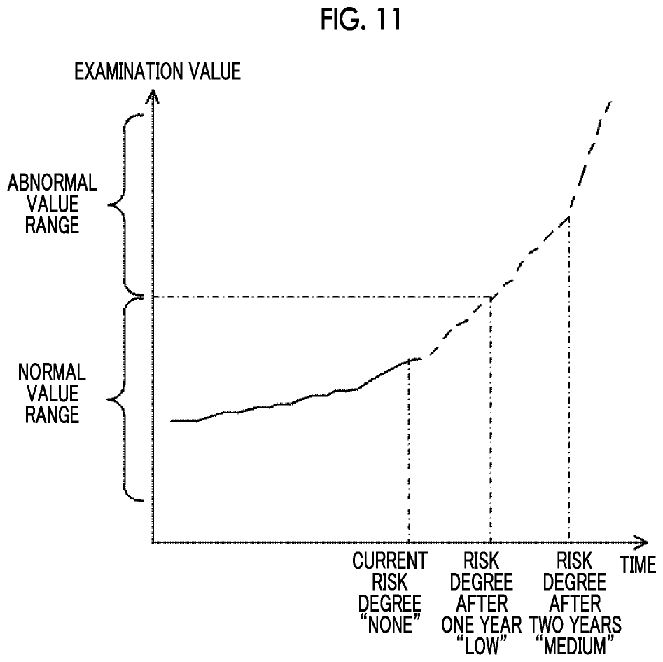

Further, in this embodiment, as shown in FIG. 11 as an example, the derivation unit 40 may derive the risk degree using not only examination values up to the present time but also a future prediction result of the examination values. A solid line in FIG. 11 represents a time-series transition of the examination values up to the present time, and a broken line represents a future prediction result of the examination values. This prediction result is predicted using, for example, the time-series transition of the examination values up to the present time and accumulated data of the same examination values of the same-breed dogs in the past.

In the example of FIG. 11, since a current risk degree is “none”, notification of a visit promotion message is not performed. On the other hand, a risk degree based on a prediction result of the examination values one year later is “low”, and a risk degree based on a prediction result of the examination values two years later is “medium”. In this example, in a case where the subject does not visit a hospital as it is, the notification of the visit promotion message is given 1 year later at a frequency according to the risk degree of “low”, and is given 2 years later at a frequency according to the risk degree of “medium”.

Further, the risk degree derivation process using this prediction result may be performed in the above embodiment. In this case, for example, a configuration in which a future total value of risk points is predicted from a time-series transition of a total value of the risk points up to the present may be used. In addition, both information obtained by examining a subject and an examination result of the subject obtained by examining the subject may be applied as the state information.

Further, in the above-described embodiment, the notification unit 44 may further notify an owner of a subject of a message for supporting a medical treatment reservation in addition to a visit promotion message. In this case, the message for supporting the medical treatment reservation may include URL (Internet Uniform Resource Locator) of a medical treatment reservation system of a veterinary hospital.

Further, in the above-described embodiment, a case where a dog is applied as an animal that is a subject has been described, but the present invention is not limited thereto. As the animal that is the subject, for example, an animal other than the dog, such as a cat, may be applied.

Further, in the above embodiment, for example, as a hardware structure of processing units that execute various processes, such as the derivation unit 40, the determination unit 42, and the notification unit 44, the following various processors may be used. As described above, the various processors include, in addition to the CPU that is a general-purpose processor that executes software (programs) to function as various processing units, a programmable logic device (PLD) that is a processor of which a circuit configuration is changeable after manufacturing, such as a field programmable gate array (FPGA), a dedicated electrical circuit that is a processor having a circuit configuration specifically designed to execute a specific process, such as an application specific integrated circuit (ASIC).

One processing unit may be configured by one of these various processors, or may be configured by a combination of two or more processors of the same type or different types (for example, a combination of a plurality of FPGAs or a combination of a CPU and an FPGA). Further, a plurality of processing units may be configured by one processor.

As an example in which the plurality of processing units is configured by one processor, first, as represented by a computer such as a client and a server, there is a configuration in which one processor is configured by a combination of one or more CPUs and software and the processor functions as a plurality of processing units. Second, as represented by a system on chip (SoC) or the like, there is a configuration in which a processor that realizes the functions of the entire system including a plurality of processing units by one integrated circuit (IC) chip is used. As described above, the various processing units are configured using one or more of the above various processors as a hardware structure.

Further, as a hardware structure of these various processors, more specifically, electric circuitry in which circuit elements such as semiconductor elements are combined may be used.

Further, in the above-described embodiment, a configuration in which the information processing program 30 is stored (installed) in the storage unit 22 in advance has been described, but the invention is not limited thereto. The information processing program 30 may be provided in a form of being recorded on a recording medium such as a compact disc read only memory (CD-ROM), a digital versatile disc read only memory (DVD-ROM), a universal serial bus (USB) memory, or the like. Further, the information processing program 30 may be downloaded from an external device through a network.

What is claimed is:

1. An information processing apparatus comprising:
a derivation unit that derives, on the basis of state information representing a state of a subject that is a basis of diagnosis of an animal that is the subject and a breed of the subject, a degree of risk that the subject is affected with a susceptible disease;
a determination unit that determines whether or not notification of a message for promoting visit to a veterinary hospital is necessary on the basis of the degree of the risk; and
a notification unit that notifies an owner of the subject of the message in a case where it is determined by the determination unit that the notification of the message is necessary.

2. The information processing apparatus according to claim 1,
wherein the notification unit notifies the owner of the subject of the message at a higher frequency as the degree of the risk is higher in a case where it is determined by the determination unit that the notification of the message is necessary.

3. The information processing apparatus according to claim 1,
wherein the derivation unit derives the degree of the risk using a future prediction result of the state information in addition to the state information up to the present.

4. The information processing apparatus according to claim 1,
wherein the state information includes at least one of information obtained by examining the subject or an examination result of the subject.

5. An information processing method executed by a computer, the method comprising:
deriving, on the basis of state information representing a state of a subject that is a basis of diagnosis of an animal that is the subject and a breed of the subject, a degree of risk that the subject is affected with a susceptible disease;
determining whether or not notification of a message for promoting visit to a veterinary hospital is necessary on the basis of the degree of the risk; and
notifying an owner of the subject of the message in a case where it is determined that the notification of the message is necessary.

6. The information processing method according to claim 5,
notifying the owner of the subject of the message at a higher frequency as the degree of the risk is higher in a case where it is determined that the notification of the message is necessary.

7. The information processing method according to claim 5,
deriving the degree of the risk using a future prediction result of the state information in addition to the state information up to the present.

8. The information processing method according to claim 5,
wherein the state information includes at least one of information obtained by examining the subject or an examination result of the subject.

9. A non-transitory recording medium recording an information processing program causing a computer to execute:
a process of deriving, on the basis of state information representing a state of a subject that is a basis of diagnosis of an animal that is the subject and a breed of the subject, a degree of risk that the subject is affected with a susceptible disease;
a process of determining whether or not notification of a message for promoting visit to a veterinary hospital is necessary on the basis of the degree of the risk; and
a process of notifying an owner of the subject of the message in a case where it is determined that the notification of the message is necessary.

10. The non-transitory recording medium according to claim 9,
notifying the owner of the subject of the message at a higher frequency as the degree of the risk is higher in a case where it is determined that the notification of the message is necessary.

11. The non-transitory recording medium according to claim 9,
deriving the degree of the risk using a future prediction result of the state information in addition to the state information up to the present.

12. The non-transitory recording medium according to claim 9,
wherein the state information includes at least one of information obtained by examining the subject or an examination result of the subject.
